(19) United States
(12) Patent Application Publication
(10) Pub. No.: US 2025/0372252 A1
(43) Pub. Date: Dec. 4, 2025

(54) AUTOMATIC REGIONAL LUNG DISEASE QUANTIFICATION FROM THORAX X-RAY IMAGES

(71) Applicant: KONINKLIJKE PHILIPS N.V., EINDHOVEN (NL)

(72) Inventors: ANDRÉ GOOSSEN, ELDENA (DE); TIM PHILIPP HARDER, AHRENSBURG (DE); SVEN KROENKE-HILLE, HAMBURG (DE); THOMAS BUELOW, GROSSHANSDORF (DE); JENS VON BERG, HAMBURG (DE); AXEL SAALBACH, HAMBURG (DE); HARALD ITTRICH, HAMBURG (DE); MICHAEL GRASS, BUCHHOLZ IN DER NORDHEIDE (DE)

(21) Appl. No.: 18/876,209

(22) PCT Filed: Jun. 27, 2023
(86) PCT No.: PCT/EP2023/067367

§ 371 Date: Dec. 18, 2024

Related U.S. Application Data
(60) Provisional application No. 63/356,664, filed on Jun. 29, 2022.

(51) Int. Cl. G16H 50/20; G16H 30/40; G16H 50/50; G16H 50/70

(52) U.S. Cl. CPC G16H 50/20; G16H 30/40; G16H 50/50; G16H 50/70

(57) ABSTRACT

Systems, apparatuses and methods provide technology to automatically evaluate diagnostic images, including receiving a diagnostic image relating to a condition of a patient, performing a registration of the diagnostic image with reference to an anatomical structure, identifying one or more ROIs from the registered image, generating a feature distribution based on the one or more ROIs, analyzing the feature distribution to determine a quantification, the quantification reflecting the condition of the patient, and providing a diagnostic output based on the quantification. In embodiments, identifying a ROI includes identifying a field of interest in the registered image, the field of interest encompassing the one or more regions of interest, and dividing the field of interest into a plurality of sub-regions. In embodiments, generating a feature distribution includes generating an intensity histogram for each ROI. In embodiments, analyzing the feature distribution includes determining one or more metrics based on the feature distribution.

### TECHNICAL FIELD

Embodiments generally relate to computing technology. More particularly, embodiments relate to automated disease quantification from thorax X-ray images.

### BACKGROUND

X-ray images are used to detect and stage multiple different lung diseases. As one example, among other diseases, COVID-19 is diagnosed and staged for hospitalized patients using, e.g., portable X-ray imaging systems, either after initial PCR diagnosis or throughout the hospitalization phase. Indeed, patients admitted to an intensive care unit (ICU) regularly receive portable X-ray images (e.g., daily, every other day, every three days, and so on) either to determine their disease stage or to check the status of their installations in more or less regular intervals. Disease evaluation using such images is mostly based on visual inspection and verbal description by a radiologist.

### SUMMARY OF THE INVENTION

There may be, therefore, a need to improve medical diagnostic imaging in terms of providing a way to automate disease quantification (such as, e.g., lung disease) from thorax X-ray images. An object of the disclosed technology is solved by the subject-matter of the appended independent claims, wherein further embodiments are incorporated in the dependent claims, in the accompanying drawings and the following description.

Disclosed herein are improved computing systems, methods, and computer readable media to automatically evaluate diagnostic images. In accordance with one or more embodiments, a computer-implemented method comprises receiving a diagnostic image relating to a condition of a patient, performing a registration of the diagnostic image with reference to an anatomical structure to form a registered image, identifying one or more regions of interest from the registered image, generating a feature distribution based on the one or more regions of interest, analyzing the feature distribution to determine a quantification, the quantification reflecting the condition of the patient, and providing a diagnostic output based on the quantification.

In accordance with one or more embodiments, a computer-implemented system comprises a processor, and a memory coupled to the processor, the memory comprising instructions which, when executed by the processor, cause the computing system to perform operations comprising receiving a diagnostic image relating to a condition of a patient, performing a registration of the diagnostic image with reference to an anatomical structure to form a registered image, identifying one or more regions of interest from the registered image, generating a feature distribution based on the one or more regions of interest, analyzing the feature distribution to determine a quantification, the quantification reflecting the condition of the patient, and providing a diagnostic output based on the quantification.

In accordance with one or more embodiments, at least one non-transitory computer readable storage medium comprises instructions which, when executed by a computing system, cause the computing system to perform operations comprising receiving a diagnostic image relating to a condition of a patient, performing a registration of the diagnostic image with reference to an anatomical structure to form a registered image, identifying one or more regions of interest from the registered image, generating a feature distribution based on the one or more regions of interest, analyzing the feature distribution to determine a quantification, the quantification reflecting the condition of the patient, and providing a diagnostic output based on the quantification.

The features, functions, and advantages of the disclosed technology can be achieved independently in various examples or can be combined in yet other examples, further details of which can be seen with reference to the following description and drawings.

### BRIEF DESCRIPTION OF THE DRAWINGS

The various advantages of the embodiments will become apparent to one skilled in the art by reading the following specification and appended claims, and by referencing the following drawings, in which:

FIG. 1 provides a block diagram illustrating an overview of an example of an automated diagnostic system according to one or more embodiments;

FIG. 2 provides a block diagram illustrating an example of an automated diagnostic system according to one or more embodiments;

FIG. 3 provides example chest X-ray images illustrating application of a contour detection step according to one or more embodiments;

FIG. 4 provides example chest X-ray images illustrating region of interest identification and isolation according to one or more embodiments;

FIGS. 5A-5D provide examples of region of interest identification and division into sub-regions according to one or more embodiments;

FIG. 6 provides an example of generating a feature distribution based on an identified region of interest according to one or more embodiments;

FIG. 7 provides an example visual display of regional diagnostic output according to one or more embodiments;

FIG. 8 provides an example visual display of a diagnostic output timeline according to one or more embodiments;

FIG. 9A-9D provide flow charts illustrating example methods relating to performing automated diagnostic image evaluation according to one or more embodiments; and

FIG. 10 is a diagram illustrating an example of a computing system for use in an automated diagnostic system according to one or more embodiments.

### DESCRIPTION OF EMBODIMENTS

Disclosed herein are improved computing systems, methods, and computer readable media to automatically evaluate diagnostic images. As described herein, technology operates to identify one or more regions of interest, generate a feature distribution for the regions of interest, analyze the feature distribution to quantify the patient's condition, and provide a diagnostic output. The disclosed technology helps improve the overall performance of diagnostic systems by providing an automatic regional quantification algorithm and user interface for patients is described which can be used for lung diseases/conditions such as, e.g., COVID-19, pneumonia, pleural effusion, pneumothorax, etc. The technology enables the derivation of quantitative values to characterize disease which can be correlated with other clinical parameters and can serve as a basis for patient staging, outcome prediction and therapy decision.

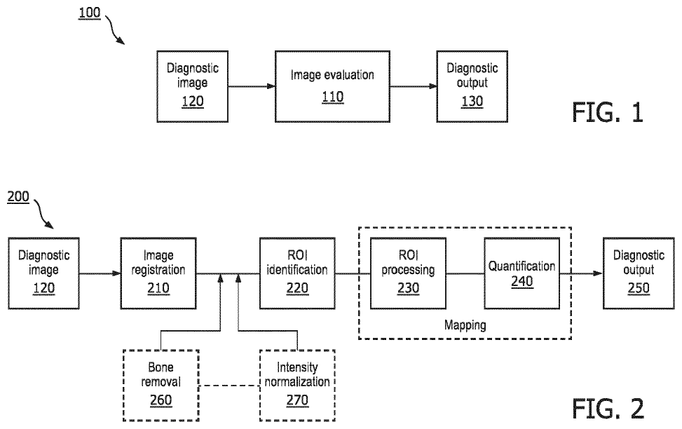

FIG. 1 provides a block diagram illustrating an overview of an example of an automated diagnostic system 100 according to one or more embodiments, with reference to components and features described herein including but not limited to the figures and associated description. As shown in FIG. 1, the diagnostic system 100 includes an image evaluation module 110 that operates to process and analyze a diagnostic image 120 and provide a diagnostic output 130. The image evaluation module 110 includes several modules (e.g., algorithms) such as image registration, region of interest (ROI) identification, ROI processing and quantification, which are described further herein with reference to FIG. 2. As so equipped, the diagnostic system 100 enables the robust derivation of comparable, quantitative numbers from variable imaging-such as, e.g., portable chest X-ray images (with their strong variability in terms of acquisition geometry, overlaying structure, patient shape and positioning). The diagnostic output 130 can include, for example, visualization such as, e.g., images (e.g., enhanced with outlining of the lung regions), features from ROI processing (e.g., histograms), quantification (e.g., scoring based on feature analysis), as well as a timeline showing progress or disease progression over time. Other diagnostic outputs are possible.

The diagnostic image 120 is an image generated by a diagnostic imaging system and can be of a variety of types or modalities. Typically, the diagnostic image 120 will include two-dimensional (2D) thorax imaging such as, e.g., chest X-ray (including upright or supine), computed tomography (CT) scanogram, forward projected CT scan, X-ray projections from image-guided therapy (IGT) systems, and/or an image obtained via other imaging techniques (such as, e.g., ultrasound). As further examples, in some embodiments the diagnostic image 120 is obtained from an X-ray system such as a conventional (absorption), dual energy/spectral (detector or tube based), or a phase contrast X-ray system. In some embodiments non-conventional imaging provides different images (e.g. soft tissue/bone image for spectral, or absorption/phase/dark field for phase contrast) that are used in one or more of the processing tasks described herein in addition to or in place of the diagnostic image 120. For example, when the diagnostic image 120 is generated from a CT system scanogram/forward projected CT, the CT system can also be or include spectral/phase contrast imaging. The diagnostic image 120 typically relates to a condition of a patient. For example, the diagnostic image 120 can be a chest X-ray relating to a condition of a patient's lungs such as, e.g., with the presence or absence of indicia of pneumonia, COVID-19, or other lung diseases/conditions including chronic obstructive pulmonary disease (COPD), tuberculosis, pleural effusion, pneumothorax, etc.

FIG. 2 provides a block diagram illustrating an example of an automated diagnostic system 200 according to one or more embodiments, with reference to components and features described herein including but not limited to the figures and associated description. In embodiments the automated diagnostic system 200 corresponds to the automated diagnostic system 100 (FIG. 1, already discussed). The diagnostic system 200 is operable to automatically evaluate the diagnostic image 120 (FIG. 1, already discussed) and provide diagnostic output (such as diagnostic output 130 in FIG. 1, already discussed). As shown in FIG. 2, the diagnostic system 200 includes an image registration module 210, a region of interest (ROI) identification module 220, a ROI processing module 230, a quantification module 240, and a diagnostic output module 250. In some embodiments, the ROI processing module 230 and the quantification module 240 are carried out via a mapping module. In some embodiments, the diagnostic system 200 includes one or more of a bone removal module 260 and/or an intensity normalization module 270.

The image registration module 210 operates to reduce or eliminate variability between different diagnostic images 120 arising, e.g., based on variations in imaging setup, acquisition geometry, overlaying structure, patient positioning, and other such variations (e.g., as typically occurring with portable X-ray units), variations in patient size/anatomy, etc. Performing image registration as described herein enhances the ability to comparatively evaluate, in an automated manner, diagnostic images from a patient taken over time, diagnostic images taken of different patients, etc. In embodiments, registration of diagnostic images 120 (e.g., portable chest X-ray images) is performed with respect to a mean standard lung image (e.g., anatomical atlas) to achieve equivalent, anatomically aligned, lung field representation. For example, in some embodiments, diagnostic image registration is done via a method based on based on the lung-contour-probability map of a trained convolution neural network (CNN) model. The method operates to automatically register the diagnostic image 120 according to aspects of (i) collimation, (ii) patient rotation, and (iii) inhalation state of a chest PA radiograph by elastic deformation of the image (e.g., image warp). Anatomical features are robustly detected by a combination of three convolutional neural networks and two probabilistic anatomical atlases. An example of such a technique described in Jens von Berg et al., “Robust chest x-ray quality assessment using convolutional neural networks and atlas regularization,” Medical Imaging 2020: Image Processing. Vol. 11313. International Society for Optics and Photonics, 2020, which is incorporated herein by reference in its entirety. In some embodiments other image registration technique(s) are employed.

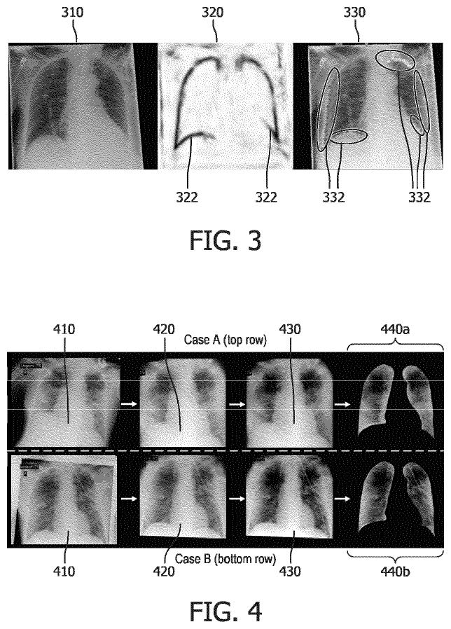

In some embodiments, an additional contour detection step is included to improve the detection of the outline of the lungs in the diagnostic image as part of the image registration process. For example, the additional contour detection is performed and the result provided as input to the atlas registration process. Performing the additional contour detection step enhances lung contour detection in images such as those produced by portable (i.e., mobile) chest X-ray systems, which are typically of lower image quality than standard chest radiographs. Referring now to FIG. 3, an example using chest X-ray images illustrates application of the additional contour detection step according to one or more embodiments, with reference to components and features described herein including but not limited to the figures and associated description. The left image shows an example diagnostic chest X-ray image 310. The middle image illustrates an inverse contour probability map 320 output by a trained CNN, where dark values correspond to high contour probability for detected lung contours 322. The right image 330 illustrates contour points 332 overlaid on the original image detected by analysis of the contour probability map 320. The contour points 332 are detected, for example, by separate CNNs, multiple separate tasks of one single CNN, or by a contour point classification task, such that it is known whether a point belongs to a certain portion of the contour. These contour points are then used to map the image to the atlas. The contour points 332 identify more precisely the lung contour, thus illustrating the enhancement provided by the additional lung contour detection.

Returning now to FIG. 2, the ROI identification module 220 operates to identify and extract or isolate, for further analysis, those region(s) of interest in the diagnostic image 120 (as registered via the image registration module 210). For example, for lung images the region(s) of interest are typically the lung field (two lungs). In embodiments the region(s) of interest are identified, e.g., based on imagery output from the image registration process. For example, in some embodiments the lung field is identified and isolated based on drawing a mask onto the atlas-mapped image and then using the mask to isolate the region(s) of interest in the registered diagnostic image. For example, in embodiments the masking is performed by a clinical expert, with computer assistance provided via a user interface. As another example, in some embodiments the lung field is identified and isolated based on the lung contours identified in the image registration process. In embodiments, these techniques are combined.

Referring now to FIG. 4, an example of chest X-ray images for a patient taken at two different times illustrates identification and isolation of the ROIs according to one or more embodiments, with reference to components and features described herein including but not limited to the figures and associated description. Diagnostic images for the patient at a first time (Case A) are in the top row, and images for the patient at a second time (Case B) are in the bottom row. For each case, the images 410 in the left column show the original diagnostic chest X-ray images. In the next column, images 420 show the result of registration of the images 410 to an anatomical atlas. The images 420 also show the result of applying an optional bone removal process. The optional bone removal module 260 is described further below. In the next column, images 430 show the result of applying an optional intensity normalization process to the images 420. The optional intensity normalization module 270 is described further below. In the right column, image 440a shows the identified and isolated lung field for the patient Case A, and image 440b shows the identified and isolated lung field for the patient Case B.

In some embodiments, a field of interest is identified that encompasses the region(s) of interest. The field of interest (region(s) of interest) is then divided into a plurality of regions (e.g., subdivided into sub-regions). In embodiments, the division of the field of interest into regions (e.g., sub-regions) is based on anatomical landmarks. For example, the lung field identified and isolated from the registered image (such as, e.g., the lung field shown in image 440a and image 440b of FIG. 4) is divided into a plurality of regions or sub-regions. In examples, the division into a plurality of regions or sub-regions is based on anatomical landmarks such as, e.g., dividing each lung into approximately comparable lung volume per region, or based on fitting a lung model (e.g., three-dimensional lung lobe models to the 2D projection image).

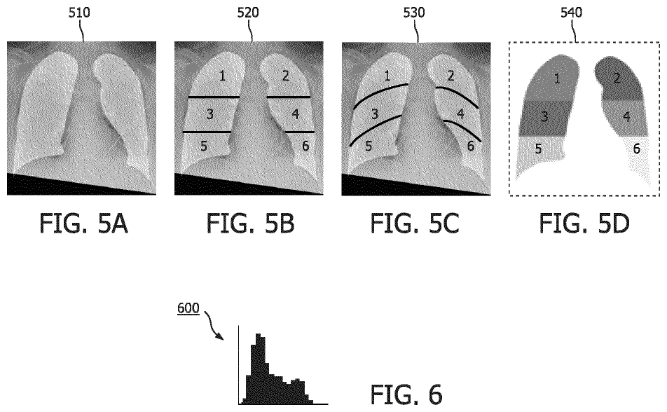

FIGS. 5A-5D illustrate examples of ROI identification and division into sub-regions according to one or more embodiments, with reference to components and features described herein including but not limited to the figures and associated description. Referring now to FIG. 5A, an example of an X-ray chest image 510 is shown with an overlay of the identified lung field (light gray overlay) as a field of interest (regions of interest). FIG. 5B illustrates one example of sub-dividing the field of interest from FIG. 5A into six (6) sub-regions, labeled 1 through 6 (image 520), based on an approximated comparable lung volume per region. FIG. 5C illustrates another example of sub-dividing the field of interest from FIG. 5A into six (6) sub-regions, labeled 1 through 6 (image 530), based on fitting a lung model. In some embodiments, the fitting is based on identifying particular ribs in the thorax from the original image. FIG. 5D illustrates a graphical depiction 540 of the sub-regions 1-6 based on the division of the lung field as shown in FIG. 5B (the shading in FIG. 5D is merely to help illustrate the areas covered by the various regions 1-6 and does not reflect a condition of the patient's lungs).

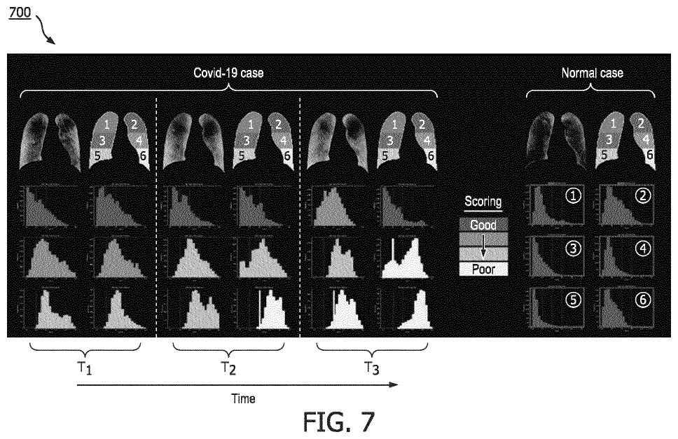

Returning now to FIG. 2, the ROI processing module 230 operates to process the region(s) of interest identified in the ROI identification module 220 to generate a feature distribution based on the region(s) of interest. As one example, generating a feature distribution can include generating a histogram of the region(s) of interest that provides a distribution of the intensity of pixels in the region(s). An example of a histogram 600 for a region of interest is illustrated in FIG. 6 according to one or more embodiments, with reference to components and features described herein including but not limited to the figures and associated description. FIG. 6 depicts a histogram of the pixel intensities (X-axis) showing the number of pixels in the ROI having a particular intensity (Y-axis). In some embodiments, where a field of interest is divided into sub-regions, a histogram is generated for each of the sub-regions. An example of histograms generated for a plurality of sub-regions is illustrated in FIG. 7 (as described further herein). In embodiments, one or more other feature distributions are generated in addition to or as an alternative to histograms. Examples of such other feature distributions include a local intensity statistical distribution (e.g., mean and standard deviation), an edge distribution (e.g., via edge detection), area estimation, etc.

Returning now to FIG. 2, the quantification module 240 operates to analyze the feature distribution to determine a quantification (e.g., a metric, score, etc.), where the quantification reflects the condition of the patient in relation to the disease or condition under consideration. As an example, in some embodiments the feature distribution includes an intensity histogram, and the quantification module 240 analyzes the resulting histogram to determine a peak position and width of the peak area. Such an analysis can be used to identify disease condition or progression. For example, if a histogram is obtained for a patient's lung field of a chest X-ray, and if the peak value of the histogram is of relatively low intensity, this tends to indicate that the lungs of the patient are relatively clear and the patient therefore has lungs in good condition. As another example, if a histogram is obtained for a patient's lung field of a chest X-ray, and if the peak value of the histogram is of medium to high intensity, this tends to indicate that the lung field of the X-ray is at least partially cloudy and the patient therefore has lungs in moderate-to-poor condition. In some embodiments, other analyses are performed on the feature distribution, such as, e.g., a multi-gaussian distribution fit, to provide a more fine-grained analysis of the feature. In some embodiments, a histogram is obtained and analyzed for each a plurality of sub-regions of the lung field, which provides a finer-grained evaluation (based on multiple histograms) than analysis of a single histogram of the lung field.

In embodiments the quantification module 240 further operates to provide a score indicating a disease condition or a stage of disease progression. The score can be based on such indicia as comparing feature analyses to similar analyses for patients of known condition, a predetermined scale (e.g., based on professional or expert experience), etc. For example, in some embodiments, where the feature distribution includes an intensity histogram and an analysis is conducted (e.g., peak location/width, multi-gaussian fit, etc.) the quantification module 240 determines a score based upon the analysis. The score includes, e.g., one or more of a numerical score indicating a severity of disease (e.g., a sore of 1-5), a qualitative rank indicating condition (e.g., a series of qualitative ranks ranging from Good to Poor), etc. In some embodiments, the score can be based on comparing a feature analysis result to one or more thresholds.

For example, where a histogram is obtained for a patient's lung field of a chest X-ray, and the peak value of the histogram is of relatively low intensity (which tends to indicate that the lungs of the patient are relatively clear and the patient therefore has lungs in good condition), a score of “GOOD” is assigned. As another example, where a histogram is obtained for a patient's lung field of a chest X-ray, and the peak value of the histogram is of high intensity and the width is relatively broad (which tends to indicate that the lung field of the X-ray is very cloudy and the patient therefore has lungs in poor condition), a score of “POOR” is assigned.

In some embodiments, the score is based on an evaluation of a plurality of feature distributions. For example, where a histogram is obtained and analyzed for each a plurality of sub-regions of the lung field, a sub-score is generated for each sub-region, and the sub-scores are combined to generate a total score. In some embodiments, a location relating to the feature is used in determining a score. For example, where a histogram is obtained and analyzed for each of a plurality of sub-regions of the lung field, each sub-region receives a sub-score, where the sub-score is weighted based on location. An example of a scoring scheme relating to COVID-19 patients is discussed in A. Borghese, “COVID-19 outbreak in Italy: experimental chest X-ray scoring system for quantifying and monitoring disease progression,” La radiologia medica (2020) [https://doi.org/10.1007/s11547-020-01200-3], which is incorporated herein by reference in its entirety. In some embodiments, a neural network is used to evaluate a feature distribution and provide a score, the neural network trained with data such as clinical parameters, radiologist ratings, etc.

The diagnostic output module 250 operates to provide an output (results) of the quantification (evaluation) process. For example, in embodiments the output/results include a diagnosis of a disease or condition is provided. As another example, in embodiments the output/results include a progression or stage of disease/condition. In embodiments, the output/results include a score indicative of a severity or progression of disease/condition. In embodiments, output/results in the form of a diagnosis, stage/progression, score, etc. are provided over time (e.g., via a timeline). The output/results provided as described herein have a number of uses such as, e.g., to perform triage of new patients and/or to track disease progression over time, etc.

In embodiments, the diagnostic output module 250 provides a visualization of output/results, including such as those identified herein e.g., diagnosis of a disease or condition, progression or stage of disease/condition, score indicative of a severity or progression of disease/condition, and/or timeline showing diagnosis, stage/progression, score, etc. over time. In embodiments, visualization includes, e.g., one or more of a visual display (e.g. a screen on a laptop, tablet, smartphone, etc.), a web-based dashboard, document printout (including electronically-generated documents), etc.

FIG. 7 provides an example visual display 700 of regional diagnostic output according to one or more embodiments, with reference to components and features described herein including but not limited to the figures and associated description. The display in FIG. 7 (e.g., a screen display) includes images and histograms for a normal (healthy) patient on the right side of the display, and a set of images and histograms for a COVID-19 patient, showing progression over several time samples, on the left/middle part of the display. The display illustrates visualization of regional statistical analysis of the grey value distribution for pixels in the lung field regions for diseased and healthy patients, including showing the distribution change due to COVID-19 disease progression.

More particularly, for the normal patient, the first row of the display (under the heading) shows identified/isolated lung field (two lungs) for the normal patient, along with a graphical illustration (similar to the graphic of FIG. 5D) showing how the lung field is divided into 6 sub-regions (numbered 1-6). Under the first row is a set of six histograms, each histogram representing an intensity histogram for one of the six sub-regions of the isolated lung field (based on the respective image portion corresponding to the sub-region). Each histogram is labeled (1)-(6), respectively, corresponding to the labeled sub-regions 1-6. The histograms illustrated in FIG. 7 are coded by a gray value indicative of a score ranging from “Good” to “Poor,” as shown to the left of the “healthy” histograms. As reflected in the histograms for the normal patient, the peak value in each histogram correlates to a relatively low intensity (score is “Good”), indicative of a healthy patient. In embodiments, a color coding (such as, e.g., Green, Yellow, Orange, Red) is used instead of a gray scale to represent scores from “Good” to “Poor.”

Turning to the left side of FIG. 7, there are images and histograms for a COVID-19 patient taken at three times (labeled T₁, T₂ and T₃ in the figure). The first two columns show an identified/isolated lung field image at time T₁ (top row) along with a graphic showing how the lung field is divided into six sub-regions. The graphic corresponds to the same graphic and same sub-division into six sub-regions as described above for the healthy patient. The next three rows show six histograms corresponding to intensity histograms for each of the six sub-regions of the COVID patient at time T₁. The histograms are arranged in the same order as shown at the right side of the figure for the healthy patient. The histograms for time T₁ show, for the various lung sub-regions, histogram evaluations ranging from “Good” (top two histograms) to relatively “Poor” (bottom two histograms), indicative of a patient with parts of the lung relatively clear but other portions negatively impacted by disease. The next two columns show, similarly, an identified/isolated lung field image at time T₂ (top row) along with the divider graphic, and then six histograms corresponding to intensity histograms for each of the six sub-regions of the COVID patient at time T₂. Notably, the histogram at bottom right for time T₂ shows relatively high intensity peak and a “Poor” score. The histograms and, hence, scoring shows a deterioration in the patient's condition between time T₁ and T₂. The next two columns likewise show an identified/isolated lung field image at time T₃ (top row) along with the divider graphic, and then six histograms corresponding to intensity histograms for each of the six sub-regions of the COVID patient at time T₃. Notably, three histogram at lower right for time T₃ show relatively high intensity peak and a “Poor” score. The histograms and, hence, scoring shows further deterioration in the patient's condition between time T₂ and T₃.

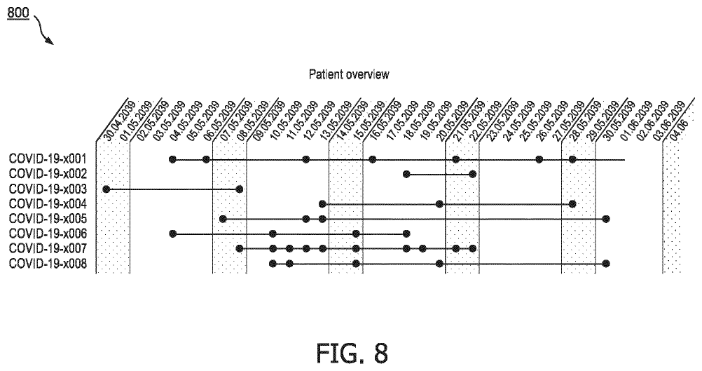

FIG. 8 provides an example visual display 800 of a diagnostic output timeline according to one or more embodiments, with reference to components and features described herein including but not limited to the figures and associated description. The display 800 (e.g., a web-based dashboard) includes a series of timelines for a group of COVID patients indicated by coded number at left of the timeline. For each patient there is a line accompanied by a series of dots indicating a result or score was automatically obtained via the diagnostic system 200 from an imaging event on a particular date (with fictitious dates shown along the top of the display). In embodiments, the dots are coded with indicia such as a number, a gray scale, a color, etc. indicative of a diagnostic score or disease stage at the particular date. Such a timeline illustrates, comparatively, disease stages (including, e.g., progression or recovery) among the various patients over time and, in embodiments, is used, e.g., to identify trends in disease progression or recovery.

Returning now to FIG. 2, in some embodiments the optional bone removal module 260 operates to remove ribs and clavicles overlaying the lung tissue in a diagnostic image after the image registration process. An example of a bone removal technique is described in Jens von Berg, “A novel bone suppression method that improves lung nodule detection,” International journal of computer assisted radiology and surgery 11.4 (2016): 641-655, which is incorporated herein by reference in its entirety. An example illustrating the results of using the bone removal technique is shown in FIG. 4, images 420 (bone removal after image registration). In some embodiments, other bone removal technique(s) are used as an alternative.

In some embodiments, the optional intensity normalization module 270 operates to obtain a standardized representation of density values on pixel grey scales. In one example, intensity normalization is performed by taking an intensity histogram of the pixel values in the lung field as well as the spine and normalizing the pixel values such that the histogram becomes essentially flat across all intensities. An example illustrating the results of using the intensity normalization technique is shown in FIG. 4, images 430 (intensity normalization after bone removal). In some embodiments, other intensity normalization technique(s) are used as an alternative. In some embodiments intensity normalization is performed after bone removal; in some embodiments intensity normalization is performed before bone removal; in some embodiments intensity normalization is performed without bone removal; in some embodiments bone removal is performed without intensity normalization. In some embodiments one or more of bone removal and/or intensity normalization is performed before ROI identification via ROI identification module 220.

While certain examples have been provided herein (including examples for diagnostic imaging relating to COVID-19 patients), the improved technology as described herein is are useful for evaluating a number of lung diseases/conditions including pneumonia, COVID-19, chronic obstructive pulmonary disease (COPD), tuberculosis, pleural effusion, pneumothorax, etc. In some embodiments, one or more modules of the diagnostic system 200 are implemented at least in part via a trained neural network (trained using data appropriate for the particular module). For example, as described herein, in some embodiments one or more trained neural networks are used for implementing at least in part the image registration module 210. Further, as described herein, in some embodiments a trained neural network is used for implementing at least in part the quantification module 240.

Some or all components in the diagnostic system 100 and/or the diagnostic system 200 can be implemented using one or more of a central processing unit (CPU), a graphics processing unit (GPU), an artificial intelligence (AI) accelerator, a field programmable gate array (FPGA) accelerator, an application specific integrated circuit (ASIC), and/or via a processor with software, or in a combination of a processor with software and an FPGA or ASIC. More particularly, components of the diagnostic system 100 and/or the diagnostic system 200 can be implemented in one or more modules as a set of program or logic instructions stored in a machine-or computer-readable storage medium such as random access memory (RAM), read only memory (ROM), programmable ROM (PROM), firmware, flash memory, etc., in hardware, or any combination thereof. For example, hardware implementations can include configurable logic, fixed-functionality logic, or any combination thereof. Examples of configurable logic include suitably configured programmable logic arrays (PLAs), FPGAS, complex programmable logic devices (CPLDs), and general purpose microprocessors. Examples of fixed-functionality logic include suitably configured ASICs, combinational logic circuits, and sequential logic circuits. The configurable or fixed-functionality logic can be implemented with complementary metal oxide semiconductor (CMOS) logic circuits, transistor-transistor logic (TTL) logic circuits, or other circuits.

For example, computer program code to carry out operations by the diagnostic system 100 and/or the diagnostic system 200 can be written in any combination of one or more programming languages, including an object oriented programming language such as JAVA, SMALLTALK, C++ or the like and conventional procedural programming languages, such as the “C” programming language or similar programming languages. Additionally, program or logic instructions might include assembler instructions, instruction set architecture (ISA) instructions, machine instructions, machine dependent instructions, microcode, state-setting data, configuration data for integrated circuitry, state information that personalizes electronic circuitry and/or other structural components that are native to hardware (e.g., host processor, central processing unit/CPU, microcontroller, etc.).

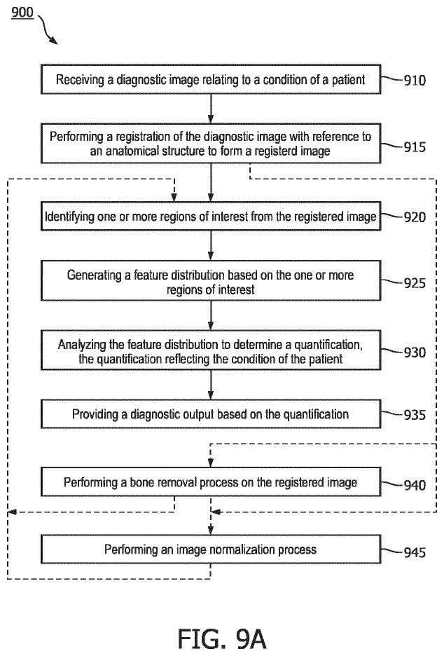

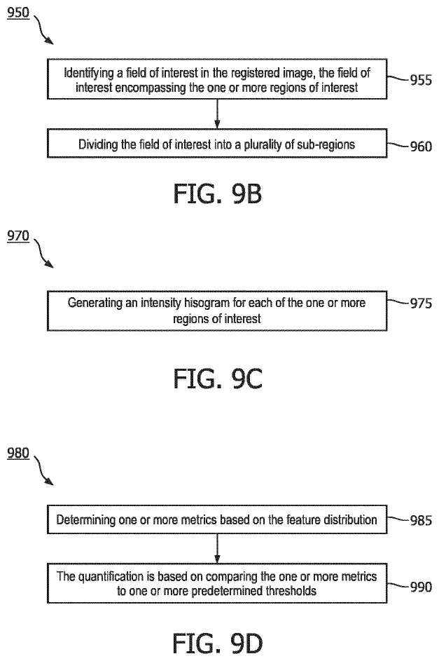

FIG. 9A provides a flow diagram illustrating an example method 900 of performing automated diagnostic image evaluation according to one or more embodiments, with reference to components and features described herein including but not limited to the figures and associated description. The method 900 can generally be implemented in the diagnostic system 100 (FIG. 1, already discussed) and/or the diagnostic system 200 (FIG. 2, already discussed). More particularly, the method 900 can be implemented as one or more modules as a set of logic instructions stored in a machine-or computer-readable storage medium such as RAM, ROM, PROM, firmware, flash memory, etc., in hardware, or any combination thereof. For example, hardware implementations can include configurable logic, fixed-functionality logic, or any combination thereof. Examples of configurable logic include suitably configured PLAs, FPGAs, CPLDs, and general purpose microprocessors. Examples of fixed-functionality logic include suitably configured ASICs, combinational logic circuits, and sequential logic circuits. The configurable or fixed-functionality logic can be implemented with CMOS logic circuits, TTL logic circuits, or other circuits.

For example, computer program code to carry out the method 900 and/or functions associated therewith can be written in any combination of one or more programming languages, including an object oriented programming language such as JAVA, SMALLTALK, C++ or the like and conventional procedural programming languages, such as the “C” programming language or similar programming languages. Additionally, program or logic instructions might include assembler instructions, instruction set architecture (ISA) instructions, machine instructions, machine dependent instructions, microcode, state-setting data, configuration data for integrated circuitry, state information that personalizes electronic circuitry and/or other structural components that are native to hardware (e.g., host processor, central processing unit/CPU, microcontroller, etc.).

Illustrated processing block 910 provides for receiving a diagnostic image relating to a condition of a patient. In embodiments the diagnostic image corresponds to the diagnostic image 120 (FIGS. 1-2, already discussed). Illustrated processing block 915 provides for performing a registration of the diagnostic image with reference to an anatomical structure to form a registered image. In some embodiments the anatomical structure relates to a lung field. In some embodiments the anatomical structure is associated with an anatomical atlas. Illustrated processing block 920 provides for identifying one or more regions of interest from the registered image. In embodiments the one or more regions of interest relate to a lung field of the patient. Illustrated processing block 925 provides for generating a feature distribution based on the one or more regions of interest. Illustrated processing block 930 provides for analyzing the feature distribution to determine a quantification, the quantification reflecting the condition of the patient. Illustrated processing block 935 provides for providing a diagnostic output based on the quantification. In some embodiments providing a diagnostic output comprises one or more of determining a diagnostic score for the condition of the patient based on the quantification, providing a visualization showing the one or more regions of interest with the feature distribution, and/or providing a visualization showing a progression of the condition of the patient over time.

In some embodiments, illustrated processing block 940 provides for performing a bone removal process on the registered image. In some embodiments, illustrated processing block 945 provides for performing an image normalization process. In some embodiments intensity normalization is performed after bone removal; in some embodiments intensity normalization is performed before bone removal; in some embodiments intensity normalization is performed without bone removal; in some embodiments bone removal is performed without intensity normalization. In some embodiments one or more of bone removal and/or intensity normalization is performed before ROI identification (block 920).

FIG. 9B provides a flow diagram illustrating an example method 950 of identifying one or more regions of interest according to one or more embodiments, with reference to components and features described herein including but not limited to the figures and associated description. In embodiments, the method 950 can be substituted for at least a portion of illustrated processing block 920 (FIG. 9A, already discussed). The method 950 can generally be implemented in the diagnostic system 100 (FIG. 1, already discussed) and/or the diagnostic system 200 (FIG. 2, already discussed). More particularly, the method 950 can be implemented as one or more modules as a set of logic instructions stored in a machine-or computer-readable storage medium such as RAM, ROM, PROM, firmware, flash memory, etc., in hardware, or any combination thereof. For example, hardware implementations can include configurable logic, fixed-functionality logic, or any combination thereof. Examples of configurable logic include suitably configured PLAs, FPGAs, CPLDs, and general purpose microprocessors. Examples of fixed-functionality logic include suitably configured ASICs, combinational logic circuits, and sequential logic circuits. The configurable or fixed-functionality logic can be implemented with CMOS logic circuits, TTL logic circuits, or other circuits.

For example, computer program code to carry out the method 950 and/or functions associated therewith can be written in any combination of one or more programming languages, including an object oriented programming language such as JAVA, SMALLTALK, C++ or the like and conventional procedural programming languages, such as the “C” programming language or similar programming languages. Additionally, program or logic instructions might include assembler instructions, instruction set architecture (ISA) instructions, machine instructions, machine dependent instructions, microcode, state-setting data, configuration data for integrated circuitry, state information that personalizes electronic circuitry and/or other structural components that are native to hardware (e.g., host processor, central processing unit/CPU, microcontroller, etc.).

Illustrated processing block 955 provides for identifying a field of interest in the registered image, the field of interest encompassing the one or more regions of interest. Illustrated processing block 960 provides for dividing the field of interest into a plurality of sub-regions.

FIG. 9C provides a flow diagram illustrating an example method 970 of generating a feature distribution according to one or more embodiments, with reference to components and features described herein including but not limited to the figures and associated description. In embodiments, the method 970 can be substituted for at least a portion of illustrated processing block 925 (FIG. 9A, already discussed). The method 970 can generally be implemented in the diagnostic system 100 (FIG. 1, already discussed) and/or the diagnostic system 200 (FIG. 2, already discussed). More particularly, the method 970 can be implemented as one or more modules as a set of logic instructions stored in a machine-or computer-readable storage medium such as RAM, ROM, PROM, firmware, flash memory, etc., in hardware, or any combination thereof. For example, hardware implementations can include configurable logic, fixed-functionality logic, or any combination thereof. Examples of configurable logic include suitably configured PLAs, FPGAs, CPLDs, and general purpose microprocessors. Examples of fixed-functionality logic include suitably configured ASICs, combinational logic circuits, and sequential logic circuits. The configurable or fixed-functionality logic can be implemented with CMOS logic circuits, TTL logic circuits, or other circuits.

For example, computer program code to carry out the method 970 and/or functions associated therewith can be written in any combination of one or more programming languages, including an object oriented programming language such as JAVA, SMALLTALK, C++ or the like and conventional procedural programming languages, such as the “C” programming language or similar programming languages. Additionally, program or logic instructions might include assembler instructions, instruction set architecture (ISA) instructions, machine instructions, machine dependent instructions, microcode, state-setting data, configuration data for integrated circuitry, state information that personalizes electronic circuitry and/or other structural components that are native to hardware (e.g., host processor, central processing unit/CPU, microcontroller, etc.).

Illustrated processing block 975 provides for generating an intensity histogram for each of the one or more regions of interest. In embodiments, the intensity histogram provides a distribution of the intensity of pixels in the one or more regions of interest.

FIG. 9D provides a flow diagram illustrating an example method 980 of analyzing the feature distribution according to one or more embodiments, with reference to components and features described herein including but not limited to the figures and associated description. In embodiments, the method 980 can be substituted for at least a portion of illustrated processing block 930 (FIG. 9A, already discussed). The method 980 can generally be implemented in the diagnostic system 100 (FIG. 1, already discussed) and/or the diagnostic system 200 (FIG. 2, already discussed). More particularly, the method 980 can be implemented as one or more modules as a set of logic instructions stored in a machine-or computer-readable storage medium such as RAM, ROM, PROM, firmware, flash memory, etc., in hardware, or any combination thereof. For example, hardware implementations can include configurable logic, fixed-functionality logic, or any combination thereof. Examples of configurable logic include suitably configured PLAs, FPGAs, CPLDs, and general purpose microprocessors. Examples of fixed-functionality logic include suitably configured ASICs, combinational logic circuits, and sequential logic circuits. The configurable or fixed-functionality logic can be implemented with CMOS logic circuits, TTL logic circuits, or other circuits.

For example, computer program code to carry out the method 980 and/or functions associated therewith can be written in any combination of one or more programming languages, including an object oriented programming language such as JAVA, SMALLTALK, C++ or the like and conventional procedural programming languages, such as the “C” programming language or similar programming languages. Additionally, program or logic instructions might include assembler instructions, instruction set architecture (ISA) instructions, machine instructions, machine dependent instructions, microcode, state-setting data, configuration data for integrated circuitry, state information that personalizes electronic circuitry and/or other structural components that are native to hardware (e.g., host processor, central processing unit/CPU, microcontroller, etc.).

Illustrated processing block 985 provides for determining one or more metrics based on the feature distribution. In some embodiments, the quantification is based on comparing the one or more metrics to one or more predetermined thresholds.

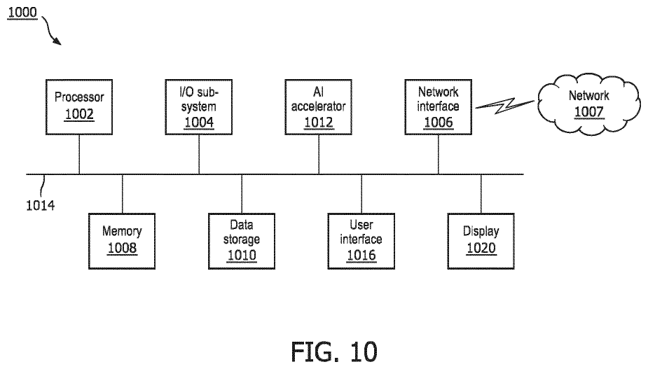

FIG. 10 is a diagram illustrating a computing system 1000 for use in the diagnostic system 100 and/or in the diagnostic system 200 according to one or more embodiments, with reference to components and features described herein including but not limited to the figures and associated description. Although FIG. 10 illustrates certain components, the computing system 1000 can include additional or multiple components connected in various ways. It is understood that not all examples will necessarily include every component shown in FIG. 10. As illustrated in FIG. 10, the computing system 1000 includes one or more processors 1002, an I/O subsystem 1004, a network interface 1006, a memory 1008, a data storage 1010, an artificial intelligence (AI) accelerator 1012, a user interface 1016, and/or a display 1020. These components are coupled, connected or otherwise in data communication via an interconnect 1014. In some embodiments, the computing system 1000 interfaces with a separate display. The computing system 1000 can implement one or more components or features of the diagnostic system 100, the diagnostic system 200, and/or any of the components or methods described herein with reference to FIGS. 1 through 9D.

The processor 1002 includes one or more processing devices such as a microprocessor, a central processing unit (CPU), a fixed application-specific integrated circuit (ASIC) processor, a reduced instruction set computing (RISC) processor, a complex instruction set computing (CISC) processor, a field-programmable gate array (FPGA), a digital signal processor (DSP), etc., along with associated circuitry, logic, and/or interfaces. The processor 1002 can include, or be connected to, a memory (such as, e.g., the memory 1008) storing executable instructions and/or data, as necessary or appropriate. The processor 1002 can execute such instructions to implement, control, operate or interface with any components or features of the diagnostic system 100, the diagnostic system 200, and/or any of the components or methods described herein with reference to FIGS. 1 through 9D. The processor 1002 can communicate, send, or receive messages, requests, notifications, data, etc. to/from other devices. The processor 1002 can be embodied as any type of processor capable of performing the functions described herein. For example, the processor 1002 can be embodied as a single or multi-core processor(s), a digital signal processor, a microcontroller, or other processor or processing/controlling circuit. The processor can include embedded instructions (e.g., processor code).

The I/O subsystem 1004 includes circuitry and/or components suitable to facilitate input/output operations with the processor 1002, the memory 1008, and other components of the computing system 1000.

The network interface 1006 includes suitable logic, circuitry, and/or interfaces that transmits and receives data over one or more communication networks using one or more communication network protocols. The network interface 1006 can operate under the control of the processor 1002, and can transmit/receive various requests and messages to/from one or more other devices. The network interface 1006 can include wired or wireless data communication capability; these capabilities can support data communication with a wired or wireless communication network, such as the network 1007, and further including the Internet, a wide area network (WAN), a local area network (LAN), a wireless personal area network, a wide body area network, a cellular network, a telephone network, any other wired or wireless network for transmitting and receiving a data signal, or any combination thereof (including, e.g., a Wi-Fi network or corporate LAN). The network interface 1006 can support communication via a short-range wireless communication field, such as Bluetooth, NFC, or RFID. Examples of network interface 1006 include, but are not limited to, one or more of an antenna, a radio frequency transceiver, a wireless transceiver, a Bluetooth transceiver, an ethernet port, a universal serial bus (USB) port, or any other device configured to transmit and receive data.

The memory 1008 includes suitable logic, circuitry, and/or interfaces to store executable instructions and/or data, as necessary or appropriate, when executed, to implement, control, operate or interface with any components or features of the diagnostic system 100, the diagnostic system 200, and/or any of the components or methods described herein with reference to FIGS. 1 through 9D. The memory 1008 can be embodied as any type of volatile or non-volatile memory or data storage capable of performing the functions described herein, and can include a random-access memory (RAM), a read-only memory (ROM), write-once read-multiple memory (e.g., EEPROM), a removable storage drive, a hard disk drive (HDD), a flash memory, a solid-state memory, and the like, and including any combination thereof. In operation, the memory 1008 can store various data and software used during operation of the computing system 1000 such as operating systems, applications, programs, libraries, and drivers. Thus, the memory 1008 can include at least one non-transitory computer readable medium comprising instructions which, when executed by the computing system 1000, cause the computing system 1000 to perform operations to carry out one or more functions or features of the diagnostic system 100, the diagnostic system 200, and/or any of the components or methods described herein with reference to FIGS. 1 through 9D. The memory 1008 can be communicatively coupled to the processor 1002 directly or via the I/O subsystem 1004.

The data storage 1010 can include any type of device or devices configured for short-term or long-term storage of data such as, for example, memory devices and circuits, memory cards, hard disk drives, solid-state drives, non-volatile flash memory, or other data storage devices. The data storage 1010 can include or be configured as a database, such as a relational or non-relational database, or a combination of more than one database. In some examples, a database or other data storage can be physically separate and/or remote from the computing system 1000, and/or can be located in another computing device, a database server, on a cloud-based platform, or in any storage device that is in data communication with the computing system 1000.

The artificial intelligence (AI) accelerator 1012 includes suitable logic, circuitry, and/or interfaces to accelerate artificial intelligence applications, such as, e.g., artificial neural networks, machine vision and machine learning applications, including through parallel processing techniques. In one or more examples, the AI accelerator 1012 can include a graphics processing unit (GPU). The AI accelerator 1012 can implement one or more components or features of the diagnostic system 100, the diagnostic system 200, and/or components or methods described herein with reference to FIGS. 1 through 9D. In some examples the computing system 1000 includes a second AI accelerator (not shown).

The interconnect 1014 includes any one or more separate physical buses, point to point connections, or both connected by appropriate bridges, adapters, or controllers. The interconnect 1014 can include, for example, a system bus, a Peripheral Component Interconnect (PCI) bus, a HyperTransport or industry standard architecture bus, a small computer system interface (SCSI) bus, a universal serial bus (USB), IIC (I2C) bus, or an Institute of Electrical and Electronics Engineers (IEEE) standard 694 bus (e.g., “Firewire”), or any other interconnect suitable for coupling or connecting the components of the computing system 1000.

The user interface 1016 includes code to present, on a display, information or screens for a user and to receive input (including commands) from a user via an input device. The display 1020 can be any type of device for presenting visual information, such as a computer monitor, a flat panel display, or a mobile device screen, and can include a liquid crystal display (LCD), a light-emitting diode (LED) display, a plasma panel, or a cathode ray tube display, etc. The display 1020 can include a display interface for communicating with the display. In some examples, the display 1020 can include a display interface for communicating with a display external to the computing system 1000.

In some examples, one or more of the illustrative components of the computing system 1000 can be incorporated (in whole or in part) within, or otherwise form a portion of, another component. For example, the memory 1008, or portions thereof, can be incorporated within the processor 1002. As another example, the user interface 1016 can be incorporated within the processor 1002 and/or code in the memory 1008. In some examples, the computing system 1000 can be embodied as, without limitation, a mobile computing device, a smartphone, a wearable computing device, an Internet-of-Things device, a laptop computer, a tablet computer, a notebook computer, a computer, a workstation, a server, a multiprocessor system, and/or a consumer electronic device. In some examples, the computing system 1000, or portion thereof, is implemented in one or more modules as a set of logic instructions stored in at least one non-transitory machine-or computer-readable storage medium such as random access memory (RAM), read only memory (ROM), programmable ROM (PROM), firmware, flash memory, etc., in configurable logic such as, for example, programmable logic arrays (PLAs), field programmable gate arrays (FPGAs), complex programmable logic devices (CPLDs), in fixed-functionality logic hardware using circuit technology such as, for example, application specific integrated circuit (ASIC), complementary metal oxide semiconductor (CMOS) or transistor-transistor logic (TTL) technology, or any combination thereof.

Embodiments of each of the above systems, devices, components and/or methods, including the diagnostic system 100, the diagnostic system 200, the method 900, the method 950, the method 970, and/or the method 980, and/or any other system components, can be implemented in hardware, software, or any suitable combination thereof. For example, hardware implementations may include configurable logic, fixed-functionality logic, or any combination thereof. Examples of configurable logic include suitably configured PLAs, FPGAs, CPLDs, and general purpose microprocessors. Examples of fixed-functionality logic include suitably configured ASICs, combinational logic circuits, and sequential logic circuits. The configurable or fixed-functionality logic can be implemented with CMOS logic circuits, TTL logic circuits, or other circuits.

Alternatively, or additionally, all or portions of the foregoing systems and/or components and/or methods can be implemented in one or more modules as a set of program or logic instructions stored in a machine-or computer-readable storage medium such as RAM, ROM, PROM, firmware, flash memory, etc., to be executed by a processor or computing device. For example, computer program code to carry out the operations of the components can be written in any combination of one or more operating system (OS) applicable/appropriate programming languages, including an object-oriented programming language such as PYTHON, PERL, JAVA, SMALLTALK, C++, C #or the like and conventional procedural programming languages, such as the “C” programming language or similar programming languages.

### Additional Notes and Examples

Example 1 includes a computer-implemented method comprising receiving a diagnostic image relating to a condition of a patient, performing a registration of the diagnostic image with reference to an anatomical structure to form a registered image, identifying one or more regions of interest from the registered image, generating a feature distribution based on the one or more regions of interest, analyzing the feature distribution to determine a quantification, the quantification reflecting the condition of the patient, and providing a diagnostic output based on the quantification.
Example 2 includes the method of Example 1, further comprising one or more of performing a bone removal process on the registered image, or performing an image normalization process.
Example 3 includes the method of Example 1 or 2, wherein identifying one or more regions of interest comprises identifying a field of interest in the registered image, the field of interest encompassing the one or more regions of interest, and dividing the field of interest into a plurality of sub-regions.
Example 4 includes the method of Example 1, 2, or 3, wherein generating a feature distribution comprises generating an intensity histogram for each of the one or more regions of interest.
Example 5 includes the method of any of Examples 1-4, wherein analyzing the feature distribution comprises determining one or more metrics based on the feature distribution.
Example 6 includes the method of any of Examples 1-5, wherein the quantification is based on comparing the one or more metrics to one or more predetermined thresholds.
Example 7 includes the method of any of Examples 1-6, wherein providing a diagnostic output comprises one or more of determining a diagnosis of diagnostic score for the condition of the patient based on the quantification, providing a visualization showing the one or more regions of interest with the feature distribution, or providing a visualization showing a progression of the condition of the patient over time.
Example 8 includes a computing system comprising a processor, and a memory coupled to the processor, the memory comprising instructions which, when executed by the processor, cause the computing system to perform operations comprising receiving a diagnostic image relating to a condition of a patient, performing a registration of the diagnostic image with reference to an anatomical structure to form a registered image, identifying one or more regions of interest from the registered image, generating a feature distribution based on the one or more regions of interest, analyzing the feature distribution to determine a quantification, the quantification reflecting the condition of the patient, and providing a diagnostic output based on the quantification.
Example 9 includes the system of Example 8, wherein the instructions, when executed, further cause the computing system to perform operations comprising one or more of performing a bone removal process on the registered image, or performing an image normalization process.
Example 10 includes the system of Example 8 or 9, wherein identifying one or more regions of interest comprises identifying a field of interest in the registered image, the field of interest encompassing the one or more regions of interest, and dividing the field of interest into a plurality of sub-regions.
Example 11 includes the system of Example 8, 9, or 10, wherein generating a feature distribution comprises generating an intensity histogram for each of the one or more regions of interest.
Example 12 includes the system of any of Examples 8-11, wherein analyzing the feature distribution comprises determining one or more metrics based on the feature distribution.
Example 13 includes the system of any of Examples 8-12, wherein the quantification is based on comparing the one or more metrics to one or more predetermined thresholds.
Example 14 includes the system of any of Examples 8-13, wherein providing a diagnostic output comprises one or more of determining diagnostic score for the condition of the patient based on the quantification, providing a visualization showing the one or more regions of interest with the feature distribution, or providing a visualization showing a progression of the condition of the patient over time.
Example 15 includes at least one non-transitory computer readable storage medium comprising instructions which, when executed by a computing system, cause the computing system to perform operations comprising receiving a diagnostic image relating to a condition of a patient, performing a registration of the diagnostic image with reference to an anatomical structure to form a registered image, identifying one or more regions of interest from the registered image, generating a feature distribution based on the one or more regions of interest, analyzing the feature distribution to determine a quantification, the quantification reflecting the condition of the patient, and providing a diagnostic output based on the quantification.
Example 16 includes the at least one non-transitory computer readable storage medium of Example 15, wherein the instructions, when executed, further cause the computing system to perform operations comprising one or more of performing a bone removal process on the registered image, or performing an image normalization process.
Example 17 includes the at least one non-transitory computer readable storage medium of Example 15 or 16, wherein identifying one or more regions of interest comprises identifying a field of interest in the registered image, the field of interest encompassing the one or more regions of interest, and dividing the field of interest into a plurality of sub-regions.
Example 18 includes the at least one non-transitory computer readable storage medium of Example 15, 16, or 17, wherein generating a feature distribution comprises generating an intensity histogram for each of the one or more regions of interest.
Example 19 includes the at least one non-transitory computer readable storage medium of any of Examples 15-18, wherein analyzing the feature distribution comprises determining one or more metrics based on the feature distribution, and wherein the quantification is based on comparing the one or more metrics to one or more predetermined thresholds.
Example 20 includes the at least one non-transitory computer readable storage medium of any of Examples 15-19, wherein providing a diagnostic output comprises one or more of determining a diagnostic score for the condition of the patient based on the quantification, providing a visualization showing the one or more regions of interest with the feature distribution, or providing a visualization showing a progression of the condition of the patient over time.
Example 21 includes an apparatus comprising means for performing the method of any one of Examples 1-7.

Embodiments are applicable for use with all types of semiconductor integrated circuit (“IC”) chips. Examples of these IC chips include but are not limited to processors, controllers, chipset components, programmable logic arrays (PLAs), memory chips, network chips, systems on chip (SoCs), SSD/NAND controller ASICs, and the like. In addition, in some of the drawings, signal conductor lines are represented with lines. Some may be different, to indicate more constituent signal paths, have a number label, to indicate a number of constituent signal paths, and/or have arrows at one or more ends, to indicate primary information flow direction. This, however, should not be construed in a limiting manner. Rather, such added detail may be used in connection with one or more exemplary embodiments to facilitate easier understanding of a circuit. Any represented signal lines, whether or not having additional information, may actually comprise one or more signals that may travel in multiple directions and may be implemented with any suitable type of signal scheme, e.g., digital or analog lines implemented with differential pairs, optical fiber lines, and/or single-ended lines.

Example sizes/models/values/ranges may have been given, although embodiments are not limited to the same. As manufacturing techniques (e.g., photolithography) mature over time, it is expected that devices of smaller size could be manufactured. In addition, well known power/ground connections to IC chips and other components may or may not be shown within the figures, for simplicity of illustration and discussion, and so as not to obscure certain aspects of the embodiments. Further, arrangements may be shown in block diagram form in order to avoid obscuring embodiments, and also in view of the fact that specifics with respect to implementation of such block diagram arrangements are highly dependent upon the platform within which the embodiment is to be implemented, i.e., such specifics should be well within purview of one skilled in the art. Where specific details (e.g., circuits) are set forth in order to describe example embodiments, it should be apparent to one skilled in the art that embodiments can be practiced without, or with variation of, these specific details. The description is thus to be regarded as illustrative instead of limiting.

The term “coupled” may be used herein to refer to any type of relationship, direct or indirect, between the components in question, and may apply to electrical, mechanical, fluid, optical, electromagnetic, electromechanical or other connections, including logical connections via intermediate components (e.g., device A may be coupled to device C via device B). In addition, the terms “first”, “second”, etc. may be used herein only to facilitate discussion, and carry no particular temporal or chronological significance unless otherwise indicated.

As used in this application and in the claims, a list of items joined by the term “one or more of” may mean any combination of the listed terms. For example, the phrases “one or more of A, B or C” may mean A, B, C; A and B; A and C; B and C; or A, B and C.

Those skilled in the art will appreciate from the foregoing description that the broad techniques of the embodiments can be implemented in a variety of forms. Therefore, while the embodiments have been described in connection with particular examples thereof, the true scope of the embodiments should not be so limited since other modifications will become apparent to the skilled practitioner upon a study of the drawings, specification, and following claims.

1. A computer-implemented method, comprising:
receiving a diagnostic image relating to a condition of a patient;
performing a registration of the diagnostic image with reference to an anatomical structure to form a registered image;
identifying one or more regions of interest from the registered image;
generating a feature distribution based on the one or more regions of interest;
analyzing the feature distribution to determine a quantification, the quantification reflecting the condition of the patient; and
providing a diagnostic output based on the quantification.

2. The method of claim 1, further comprising one or more of:
performing a bone removal process on the registered image; or
performing an image normalization process.

3. The method of claim 1, wherein identifying one or more regions of interest comprises:
identifying a field of interest in the registered image, the field of interest encompassing the one or more regions of interest; and
dividing the field of interest into a plurality of sub-regions.

4. The method of claim 1, wherein generating a feature distribution comprises generating an intensity histogram for each of the one or more regions of interest.

5. The method of claim 4, wherein analyzing the feature distribution comprises determining one or more metrics based on the feature distribution.

6. The method of claim 5, wherein the quantification is based on comparing the one or more metrics to one or more predetermined thresholds.

7. The method of claim 1, wherein providing a diagnostic output comprises one or more of:
determining a diagnostic score for the condition of the patient based on the quantification;
providing a visualization showing the one or more regions of interest with the feature distribution; or
providing a visualization showing a progression of the condition of the patient over time.

8. A computing system comprising:
a processor; and
a memory coupled to the processor, the memory comprising instructions which, when executed by the processor, cause the computing system to perform operations comprising:
receiving a registered image including a registration of a diagnostic image with reference to an anatomical structure;
identifying one or more regions of interest from the registered image;
identifying a field of interest in the registered image, the field of interest encompassing one or more identified regions of interest;
dividing the field of interest into a plurality of sub-regions;
generating a feature distribution based on the one or more regions of interest;
analyzing the feature distribution to determine a quantification, the quantification reflecting the condition of the patient; and
providing a diagnostic output based on the quantification.

9. The system of claim 8, wherein the instructions, when executed, further cause the computing system to perform operations comprising one or more of:
performing a bone removal process on the registered image; or
performing an image normalization process.

10. (canceled)

11. The system of claim 8, wherein generating a feature distribution comprises generating an intensity histogram for each of the one or more regions of interest.

12. The system of claim 11, wherein analyzing the feature distribution comprises determining one or more metrics based on the feature distribution.

13. The system of claim 12, wherein the quantification is based on comparing the one or more metrics to one or more predetermined thresholds.

14. The system of claim 8, wherein providing a diagnostic output comprises one or more of:
determining a diagnostic score for the condition of the patient based on the quantification;
providing a visualization showing the one or more regions of interest with the feature distribution; or
providing a visualization showing a progression of the condition of the patient over time.

15. At least one non-transitory computer readable storage medium comprising instructions which, when executed by a computing system, cause the computing system to perform operations comprising:
identifying a field of interest in a registered image, wherein the registered image includes a registration of a diagnostic image with reference to an anatomical structure, wherein the field of interest encompasses the one or more regions of interest;
dividing the field of interest into a plurality of sub-regions;
generating a feature distribution based on the one or more regions of interest;
analyzing the feature distribution to determine a quantification, the quantification reflecting the condition of the patient; and
providing a diagnostic output based on the quantification.

16. The at least one non-transitory computer readable storage medium of claim 15, wherein the instructions, when executed, further cause the computing system to perform operations comprising one or more of:
performing a bone removal process on the registered image; or
performing an image normalization process.

17. (canceled)

18. The at least one non-transitory computer readable storage medium of claim 15, wherein generating a feature distribution comprises generating an intensity histogram for each of the one or more regions of interest.

19. The at least one non-transitory computer readable storage medium of claim 18, wherein analyzing the feature distribution comprises determining one or more metrics based on the feature distribution, and wherein the quantification is based on comparing the one or more metrics to one or more predetermined thresholds.

20. The at least one non-transitory computer readable storage medium of claim 15, wherein providing a diagnostic output comprises one or more of:
determining a diagnostic score for the condition of the patient based on the quantification;
providing a visualization showing the one or more regions of interest with the feature distribution; or
providing a visualization showing a progression of the condition of the patient over time.
